(19) United States
(12) Patent Application Publication
(10) Pub. No.: US 2016/0095925 A1
(43) Pub. Date: Apr. 7, 2016

(54) STABLE FORMULATION OF AZACITIDINE OR SALTS THEREOF AND THEIR PROCESS FOR PREPARATION

(71) Applicant: CADILA HEALTHCARE LIMITED, Ahmedabad (IN)

(72) Inventors: Manish Chawla, Ahmedabad (IN); Narendra Soni, Ahmedabad (IN); Kashyap Nagariya, Ahmedabad (IN); Sanjay Ghanshyambhai Patel, Ahmedabad (IN)

(21) Appl. No.: 14/872,263

(22) Filed: Oct. 1, 2015

(30) Foreign Application Priority Data
Oct. 1, 2014 (IN) 3129/MUM/2014

(51) Int. Cl. A61K 47/26; A61K 31/706

(52) U.S. Cl. CPC A61K 47/26; A61K 31/706

(57) ABSTRACT

The present invention relates to a stable pharmaceutical composition comprising azacitidine or its pharmaceutically acceptable salts, one or more sugar alcohols and one or more pharmaceutically acceptable excipients, wherein said composition is suitable for parenteral administration. The invention also relates to processes for preparing the preparation of such reconstitutable formulations. The invention also relates to therapeutic methods of use of such formulations and to use of such formulations in the manufacture of medicaments.

### FIELD OF THE INVENTION

The present invention relates to a stable pharmaceutical formulation comprising azacitidine or its pharmaceutically acceptable salts. Even more particularly, the invention relates to such formulations that are prepared as powders for reconstitution in an aqueous carrier prior to parenteral administration. The invention also relates to processes for the preparation of such reconstitutable formulations. The invention also relates to therapeutic methods of use of such formulations and to use of such formulations in the manufacture of medicaments.

### BACKGROUND OF THE INVENTION

Azacitidine, which is a compound represented by the following Formula I, is referred to as “4-amino-1-β-D-ribofuranosyl-1,3,5-triazin-2(1H)-one”. A white to off-white solid, which is insoluble in acetone, ethanol, and methyl ethyl ketone; slightly soluble in ethanol/water (50/50), propylene glycol, and polyethylene glycol; sparingly soluble in water, water saturated octanol, 5% dextrose in water, N-methyl-2-pyrrolidone, normal saline, and 5% Tween 80 in water; and soluble in dimethylsulfoxide (DMSO).

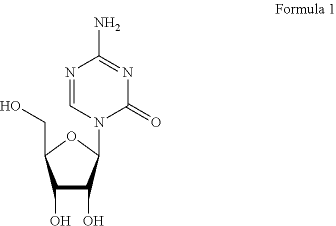

Azacitidine is believed to exert its antineoplastic effects by causing hypomethylation of DNA and direct cytotoxicity on abnormal hematopoietic cells in the bone marrow. Hypomethylation may restore normal functions to genes that are critical for differentiation and proliferation. The cytotoxic effects of azacitidine cause the death of rapidly dividing cells, including cancer cells that are no longer responsive to normal growth control mechanism. Non-proliferating cells are relatively insensitive to azacitidine. A commercially available product containing azacitidine is sold as VIDAZA®, 5-azacytidine for injection, by Celgene. The VIDAZA® product received marketing approval in the U.S. in 2004 and is supplied in a sterile form for reconstitution as a suspension for subcutaneous injection, or reconstitution as a solution with further dilution for intravenous infusion. Vials of the VIDAZA® product contain 100 mg of azacitidine and 100 mg of mannitol, as a sterile lyophilized powder.

5-Azacytidine is approved for subcutaneous (SC) or intravenous (IV) administration to treat various proliferative disorders. The s-triazine ring of 5-azacytidine has a particular sensitivity to water. See, e.g., Beisler, J. Med. Chem., 1978, 21(2), 204-08; Chan, et al., J. Pharm. Sci., 1979, 68(7), 807-12. Azacitidine rapidly degrades in aqueous solution via hydrolysis. (In an aqueous environment both in vivo and in vitro, 5-azacytidine underwent a spontaneous hydrolysis and resulted in an equilibration with a labile product, n-formylguanyl-ribosylurea, and finally the irreversible formation of guanyl-ribosylurea). Due to this instability, an aqueous formulation was not a viable option. Thus, a lyophilized dosage form was developed to minimize water activity in the medicinal product. To minimize azacitidine degradation during product manufacturing, the manufacturing process was developed such that compounding, filtration and filling operations are performed as a continuous process at reduced temperatures.

U.S. Pat. No. 4,684,630 discloses a method of parenterally delivering the aqueous-unstable 5-azacytosine arabinoside and 5-azacytidine compounds, involving an aqueous dilution of a stable, anhydrous organic solution having the drug dissolved therein. The resulting organic-aqueous solution is physiologically suitable for parenteral deliver into a warm-blooded mammal and contains the drug in an effective dosage concentration per unit volume.

U.S. Pat. No. 4,983,586 relates to aqueous parenteral solutions of drugs that are insoluble or only sparingly soluble in water, and/or unstable in water, the aqueous solutions containing hydroxypropyl-beta-cyclodextrin, to provide a means for alleviating problems associated with drug precipitation at the injection site and/or in the lungs or other organs following parenteral administration.

U.S. Pat. No. 6,943,249 relates to methods for isolating crystalline polymorphic Form I of 5-azacytidine, substantially free of other forms, and compositions thereof.

U.S. Patent Application Publication No. 2006/0128654 relates to pharmaceutical formulations of cytidine analogs and derivatives, such as 5-azacytidine, 5-aza-2′-deoxy-2′,2′-difluorocytidine, 5-aza-2′deoxy-2′-fluorocytidine, 2′-deoxy-2′,2′-difluorocytidine, and cytosine 1-β-D-arabinofuranoside, as well as methods of manufacturing the formulations. In particular, a cytidine analog or derivative is formulated with a cyclodextrin compound to stabilize and/or enhance solubility of the drug. Kits and methods for using the pharmaceutical formulations are also provided, including methods of administering the cytidine analog or derivative to treat conditions or diseases, such as cancer and hematological disorders.

U.S. Patent Application Publication No. 2006/0063735 relates to salts of 5-azacytidine as well as methods for synthesizing the salts. Pharmaceutical compositions and methods of using the 5-azacytidine salts are also provided, including methods of administering the salts or pharmaceutical compositions thereof to treat conditions such as cancer and hematological disorders.

PCT publication no. WO2013117969 discloses a process of preparing a stable pharmaceutical composition of compounds which are susceptible to hydrolysis comprising: a) Addition of required quantity of pharmaceutically acceptable lyophilization excipients optionally in Water for injection in a formulation vessel; b) Addition of organic solvent to form an appropriate proportion of aqueous and organic solvent; c) Maintaining the temperature of the formulation vessel from the range −5±1° C. to −5±3° C.; d) Addition of required quantity of compound susceptible to hydrolysis to form a solution and lyophilizing the solution.

The literature reports that VIDAZA, reconstituted with 4 mL of sterile water for injection to form a suspension for subcutaneous administration, may be stored for up to 1 hour at 25° C., or for up to 8 hours between 2° C. to 8° C. VIDAZA reconstituted with 10 mL of sterile water for injection for intravenous administration may be stored at 25° C., but the administration must be completed within 1 hour after reconstitution. The duration of IV infusion administration is limited by the decomposition and instability of azacitidine, and low aqueous solubility of the drug in aqueous solutions.

Further, azacitidine hydrolyzes quickly in water, converting into other forms, and this is dependent on pH and temperature. It has been observed that, due to hydrolysis, around nine solid state forms have been identified i.e. five polymorphic forms, three pseudo-polymorphic forms and an amorphous form. Polymorphism could be of importance since speed of dissolution of azacitidine could affect its degradation. Azacitidine rapidly degrades in aqueous solutions via hydrolysis, and due to this instability a lyophilized dosage form was developed to minimize water activity in the dosage form. Therefore, the water content of a formulation may impact the stability of the product.

Further, the lyophilized composition of azacitidine may contain degradation products that may occur during manufacturing of the drug substance and/or during the lyophilization process to make the finished drug product. Moreover, reconstitution of the lyophilized powder is difficult and the reconstitution time depends on the solvent used during lyophilization and the manufacturing parameters. Reports from clinical experience indicate that reconstitution can require at least fifteen minutes and may require as long as thirty minutes. In addition to being troublesome ad time-consuming for the healthcare professional responsible for reconstituting the product, the lengthy exposure of azacitidine to water during the reconstitution process increases the potential for loss of potency and impurity formation, due to hydrolysis of the product by water.

Hence, there remains a need for stable composition of azacitidine or its pharmaceutically acceptable salts having better impurity profile over the current commercial lyophilized compositions of azacitidine, and having simple and cost-effective methods of preparation.

### SUMMARY OF THE INVENTION

In one general aspect, the present invention relates to a stable pharmaceutical composition comprising azacitidine or its pharmaceutically acceptable salts, one or more sugar alcohols and one or more pharmaceutically acceptable excipients, wherein said composition is suitable for parenteral administration.

In another general aspect, the present invention relates to a stable pharmaceutical composition suitable for parenteral administration comprising azacitidine or its pharmaceutically acceptable salts, one or more sugar alcohols and one or more pharmaceutically acceptable excipients, wherein said composition is not lyophilized.

In another general aspect, the present invention relates to a stable pharmaceutical composition suitable for parenteral administration comprising azacitidine or its pharmaceutically acceptable salts, one or more sugar alcohols and one or more pharmaceutically acceptable excipients, wherein said composition is a reconstitutable formulation.

In another general aspect, the present invention relates to a kit comprises a stable reconstitutable pharmaceutical composition suitable for parenteral administration comprising azacitidine or its pharmaceutically acceptable salts, one or more sugar alcohols and one or more pharmaceutically acceptable excipients, and a reconstitution vehicle.

In another general aspect, the present invention relates to a process of preparing a stable pharmaceutical composition suitable for parenteral administration comprising the following steps:
a) sifting azacitidine or its pharmaceutically acceptable salts and sugar alcohols in a suitable containers;
b) sifting both the powders of step a) together with geometric mixing through a #100 ASTM SS sieve;
c) sterilizing the powder blend of step b) by gamma radiation; and,
d) filling the sterilized powder of step c) in glass vials and sealing.

In another general aspect, the present invention relates to a method for the treatment of myelodysplastic syndrome comprising a stable pharmaceutical composition suitable for parenteral administration comprising azacitidine or its pharmaceutically acceptable salts, one or more sugar alcohols and one or more pharmaceutically acceptable excipients, wherein said composition is a reconstitutable formulation.

Embodiment of the method of treatment for myelodysplastic syndrome may include particularly, refractor anemia (RA) or refractory anemia with ringed sideroblasts (if accompanied by neutropenia or thrombocytopenia or requiring transfusions), refractory anemia with excess blasts (RAEB), refractory anemia with excess blasts in transformation (RAEB-T), and chronic myelomonocytic leukemia.

Additional objects and advantages of the invention will be set forth in part in the description which follows, and in part will be obvious from the description, or may be learned by practice of the invention.

### DETAILED DESCRIPTION OF THE INVENTION

In an embodiment of the present invention, provides a stable pharmaceutical formulation comprising azacitidine or its pharmaceutically acceptable salts, which is susceptible to hydrolysis. Further, provides the invention includes the methods of using the formulations for treating various cancerous diseases.

The pharmaceutical formulations as developed by the inventors of the present invention are provided as dr powder that is suitable for parenteral administration after reconstitution with a suitable diluting fluid.

The term “stable” in the present invention is referred as to both the physical and chemical stability.

The term “pharmaceutically acceptable” refers to an ingredient that is useful in preparing a pharmaceutical composition that is generally safe, non-toxic, and neither biologically nor otherwise undesirable, and includes those acceptable for veterinary use as well as human pharmaceutical use.

The term “formulation or composition” in accordance with the present invention refers to any of various dosage forms suitable for administration of a drug, such as parenterally, intravenously, intraarterially, intramuscularly, subcutaneously etc.

The term “Azacitidine” is indented to include the free base as well as salts, polymorphs, isomers, enantiomers, hydrates, prodrugs, and any mixtures thereof.

Azacitidine is susceptible to hydrolysis and hydrolyzes quickly in water. In aqueous solution it is attacked by water molecules via neutrophilic reaction as a result of this hydrolytic cleavage, N-Formyl compound hydrolysis product “RGU-CHO” is formed which is reversible reaction and compounds are in equilibrium with each other. This impurity is formed very quickly in aqueous solution. “RGU-CHO” is then later converted to impurity “RGU” by the loss of formic acid which is an irreversible reaction. RGU-CHO is N-(formylamidino) N′-β-D-ribofuranosyl urea and RGU is I-P-D-ribofuranosyl-3-guanylurea

Conversion of Azacitidine to RGU-CHO in water is temperature dependent and directly proportional to temperature. Conversion of RGU-CHO to RGU is temperature and pH dependent. At higher temperature and in acidic and basic conditions (optimal pH for stable formulation is 6-7) formation of RGU is increased.

Therefore, the inventors of present invention provides a stable pharmaceutical composition comprising azacitidine or its pharmaceutically acceptable salts, one or more sugar alcohols and one or more pharmaceutically acceptable excipients, wherein said composition is suitable for parenteral administration.

In another embodiment, the present invention provides a stable pharmaceutical composition suitable for parenteral administration comprising azacitidine or its pharmaceutically acceptable salts, one or more sugar alcohols and one or more pharmaceutically acceptable excipients, wherein said composition is not lyophilized.

Azacitidine is prone to thermal degradation in aqueous media therefore it is supplied as sterile lyophilized powder or cake. Manufacture of a sterile lyophilized product necessitates that the product is usually first manufactured as a solution, sterilized by filtration, aseptically filled, and finally lyophilized to remove the solvents. All of these unit operations require that the product be held in the solution state for a defined period of time, at least for 4 hours. And thus the prolonged contact of azacitidine with aqueous solvents and then drying conducted at high temperature conditions increases the degradation and hence the impurity levels.

In another embodiment, the present invention provides a stable pharmaceutical composition suitable for parenteral administration comprising azacitidine or its pharmaceutically acceptable salts, one or more sugar alcohols and one or more pharmaceutically acceptable excipients, wherein said composition is a reconstitutable formulation.

The sugar alcohols that may be used according to the present invention, may be in any stereoisomers or geometric isomers or/and optical isomers forms. The sugar alcohol may be straight chain or cyclic in nature. The straight chain sugar alcohols may be trihydric to hexahydric sugar alcohol. Examples of the straight chain sugar alcohols include, but are not limited to, hexitols such as sorbitol, mannitol, glycerol, allitol, talitol, glucitol, iditol and dulcitol; pentitols such as xylitol, adonitol (also called ribitol) and arabitol; tetritols such as erythritol and threitol; and triols such as glyceol. Examples of cyclic sugar alcohol that may be used in the present invention include, but are not limited to, inositol and the like.

In one preferred embodiment of the present invention, the composition comprises mannitol as the sugar alcohol. The concentration of mannitol used in the composition may range from about 0.1% weight to about 60% weight of the composition. Preferably, about 40% weight to about 50% weight of the composition.

In one preferred embodiment of the present invention, provides a kit comprises a stable reconstitutable pharmaceutical composition suitable for parenteral administration comprising azacitidine or its pharmaceutically acceptable salts, one or more sugar alcohols and one or more pharmaceutically acceptable excipients, and a reconstitution vehicle.

A kit according to the present invention comprises a container holding the drug composition, a sterile reconstitution vehicle, and a sterile syringe.

The suitable reconstitution vehicle referred to in the invention is preferably aqueous based. The pharmaceutically acceptable carrier or vehicle referred to in this invention is one that has no unacceptably injurious or toxic effect on the subject when administered as a component of a composition by parenteral administration in an amount required herein. Example of suitable reconstitution vehicle includes 5% Dextrose Injection, Lactated Ringer's and Dextrose Injection, Sterile Water for injection, and the like. In another embodiment of the present invention, provides a process of preparing a stable pharmaceutical composition suitable for parenteral administration comprising the following steps:
a) sifting azacitidine or its pharmaceutically acceptable salts and sugar alcohols in a suitable containers;
b) sifting both the powders of step a) together with geometric mixing through a #100 ASTM SS sieve;
c) sterilizing the powder blend of step b) by gamma radiation; and,
d) filling the sterilized powder of step c) in glass vials and sealing.

In another embodiment of the present invention, provides a method for the treatment of myelodysplastic syndrome comprising a stable pharmaceutical composition suitable for parenteral administration comprising azacitidine or its pharmaceutically acceptable salts, one or more sugar alcohols and one or more pharmaceutically acceptable excipients, wherein said composition is a reconstitutable formulation.

The invention is further illustrated by the following examples which are provided to be exemplar of the invention and do not limit the scope of the invention.

### Example 1

### Composition

| Ingredients | Quantity (mg/vial) |
|---|---|
| Azacitidine | 100 mg |
| Mannitol (PF grade) | 100 mg |

### Process of Preparation:

a) Sifting azacitidine or its pharmaceutically acceptable salts and sugar alcohols in a suitable containers;
b) Sifting both the powders of step a) together with geometric mixing through a #100 ASTM SS sieve;
c) Sterilizing the powder blend of step b) by gamma radiation; and,
d) Filling the sterilized powder of step c) in glass vials and sealing.

TABLE 1
Analytical results of Azacitidine (API)
Azacitidine (API)

| Sr. | | | Gamma |
|---|---|---|---|
| No. | Parameters | Untreated | radiated |
| 1 | Description | White to off | White to off |
| | | white powder | white powder |
| 2 | Related Substances | | |
| | Single max. impurity | 0.04% | 0.05% |
| | Total impurities | 0.19% | 0.24% |
| 3 | Sterility | Non-Sterile | Sterile |

TABLE 2
Analytical results of Azacitidine
for Injection 100 mg and VIDAZA ®

| | | Azacitidine for | | | VIDAZA ® | |
|---|---|---|---|---|---|---|
| Product | | Injection 100 mg | | | | At |
| Name | | | At 40° C./ | | | 40° C./ |
| Sr | Param- | | 75% RH | | | 75% RH |
| No. | eters | Initial | 1 month | 3 month | Initial | 1 month |
| 1. | De- | Whte to | White to | White to | White to | White to |
| | scription | off-white | off-white | off-white | off-white | off-white |
| | | powder | powder | powder | powder | powder |
| 2. | Related Substances | | | | | |
| | Single | 0.05 | 0.03 | 0.04 | 3.03 | 0.7 |
| | max. | | | | | |
| | impurity | | | | | |
| | Total | 0.11 | 0.08 | 0.86 | 4.51 | 1.86 |
| | impurities | | | | | |

While the invention has been described in terms of its specific embodiments, certain modifications and equivalents will be apparent to those skilled in the art and are intended to be included within the scope of the invention.

We claim:

1. A stable pharmaceutical composition comprising azacitidine or its pharmaceutically acceptable salts, one or more sugar alcohols and one or more pharmaceutically acceptable excipients, wherein said composition is suitable for parenteral administration.

2. The stable pharmaceutical composition of claim 1, wherein said composition is not lyophilized.

3. The stable pharmaceutical composition of claim 1, wherein said composition is a reconstitutable formulation.

4. The stable pharmaceutical composition of claim 1, wherein the sugar alcohol comprises one or more of sorbitol, mannitol, glycerol, allitol, talitol, glucitol, iditol, dulcitol, xylitol, adonitol, ribitol, arabitol, erythritol, threitol, glycerol, and inositol.

5. The stable pharmaceutical composition of claim 4, wherein the sugar alcohol is mannitol.

6. The stable pharmaceutical composition of claim 5, wherein the mannitol is present in an amount of about 0.1% weight to about 60% weight of the composition.

7. A kit comprising a stable reconstitutable pharmaceutical composition suitable for parenteral administration comprising azacitidine or its pharmaceutically acceptable salts, one or more sugar alcohols and one or more pharmaceutically acceptable excipients, and a reconstitution vehicle.

8. The kit of claim 7, wherein the kit comprises a container holding the formulation, a sterile reconstitution vehicle, and a sterile syringe.

9. The kit of claim 8, wherein the reconstitution vehicle comprises one or more of a 5% Dextrose injection, Lactated Ringer's and Dextrose injection, and Sterile Water for Injection.

10. A process for preparing a sable pharmaceutical composition comprising the following steps:
a) sifting azacitidine or its pharmaceutically acceptable salts and sugar alcohols in suitable containers;
b) sifting both the powders of step a) together with geometric mixing through a #100 ASTM SS sieve;
c) sterilizing the powder blend of step b) by gamma radiation; and
d) filling the sterilized powder of step c) in glass vials and sealing.

11. A method of treating myelodysplastic syndrome, the method comprising administering the stable pharmaceutical composition of claim 1.

12. The composition of claim 1, wherein the said composition when stored in an accelerated stability conditions shows not more than 0.05% of any single impurity and not more than 1% of the total impurities.
